(19) United States
(12) Patent Application Publication
(10) Pub. No.: US 2008/0274215 A1
(43) Pub. Date: Nov. 6, 2008

(54) COMPOSITION FOR TREATMENT OF COLD AND FLU

(75) Inventor: Jarret Morrow, Boca Raton, FL (US)

Correspondence Address: ROBERT M. SCHWARTZ, P.A., P.O. BOX 221470, HOLLYWOOD, FL, 33022, US

(73) Assignee: NORTHERN HOLDINGS INC., West Palm Beach, FL (US)

(21) Appl. No.: 12/114,863

(22) Filed: May 5, 2008

Related U.S. Application Data
(60) Provisional application No. 60/915,711, filed on May 3, 2007.

(51) Int. Cl. A61K 36/258; A61P 31/00

(52) U.S. Cl. 424/728

(57) ABSTRACT

The present invention is a composition delivering therapeutically effective amounts of Andrographis Paniculata standardized to 5.6% of Andrographolide:Siberian Ginseng:American Ginseng:Vitamin C in a single dosage unit.

### INDEX TO RELATED APPLICATION

This application claims the benefit of U.S. Provisional Patent Application Ser. No. 60/915,711 filed May 3, 2007, the disclosure of which is incorporated by reference in its entirety.

### BRIEF SUMMARY OF THE INVENTION

The present invention relates to a novel combination of Andrographis Paniculata, Siberian Ginsing, American Ginsing, and Vitamin C. The dosage form provides greater efficacy than previous combinations and products.

Andrographis paniculata (AP) also known commonly as “King of Bitters,” is a member of the plant family Acanthaceae, and has been used for centuries in Asia to treat GI tract and upper respiratory infections, fever, herpes, sore throat, and a variety of other chronic and infectious diseases. It grows abundantly in southeastern Asia: India (and Sri Lanka), Pakistan and Indonesia—and is cultivated extensively in China and Thailand. The leaves contain the highest amount of andrographolide (AG) (2.39%), the most medicinally active phytochemical in the plant. Andrographolide has the following structural formula:

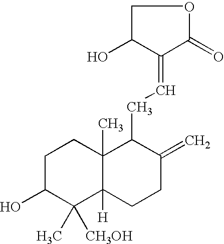

AG is also known by the chemical name 3-[2-[decahydro-6-hydroxy-5-(hydroxymethyl)-5,8a-dimethyl-2-methylene-1-napthalenyl]ethylidene]dihydro-4-hydroxy-2(3H)-furanone. It is sparingly soluble in water.

One particular commercial preparation of AP, Kang Jang®, is provided as 500 mg capsule and recommends a dosage of 2 capsule three times a day. Nature's Way® has a 300 mg dosage with recommended twice daily dosage. Nutraceuticals Science Institute® has a 1200 mg dosage form.

Siberian Ginseng or Eleutherococcus senticosus, is also known as Eleuthero. Eleuthero is the new name for Siberian ginseng in the United States. It is different from both American ginseng and Panax ginseng. They are not interchangeable. Eleuthero is a spiny-stemmed shrub with numerous stalks arising from the root, growing to a height of 9 feet in 7-10 years. It is found in northeast Asia, including much of the far southeastern Russia, northeast China, adjacent Korea, and Japan.

The constituents in Eleuthero that have received the most attention are the Eleutherosides. Seven primary Eleutherosides have been identified, with most of the research attention focusing on Eleutherosides B and E. Eleuthero also contains complex polysaccharides (a kind of sugar molecule). These constituents play a critical role in Eleuthero's ability to support immune function.

Dosages of Eleuthero are usually based on the severity of the symptoms and the type of problem. Low dosages are usually 1.0-2 grams/day—and High dosages: 9.0-15 grams/day with the Avg. dosage for Siberian ginseng usage being 2 to 6 grams a day.

American Ginseng (Panax quinquefolium) is a species of ginseng native to eastern North America. It is a perennial dicot of the family Araliaceae. Phytochemists have isolated Saponin glycosides, or Saponins, in American Ginseng. These are the primary biologically-active components of the American Ginseng root.

Low dosages of Panax quinquefolium are usually 0.5-1.0 grams/day—and High dosages: 3.0-4.5 grams/day with the average dosage for ginseng usage being 1 to 2 grams a day.

Vitamin C or L-ascorbate also has the chemical name 2-oxo-L-threo-hexono-1,4-lactone-2,3-enediol. It has a structural formula of:

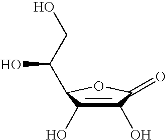

The U.S. daily recommended dosage is 75-100 mg/day.

Each of these substances individually provides beneficial therapy for colds and flu. A combination product would help preventing, treating and ameliorating the systems of colds and flu from a variety of treatment regimens. Unfortunately, the recommended dosage amounts appear to be prohibitive for providing a single, solid oral dosage product.

The present invention addresses this need by providing a single unit dosage form wherein Andrographis Paniculata, Siberian Ginseng, American Ginseng, and Vitamin C in certain ratios and dosage levels provide the desired therapy in a single, solid oral dosage form.

This is advantageous because it becomes less expensive to provide the desired therapy and a single dosage form increases patient compliance with the therapy regimen.

In one embodiment the ratio of Andrographis paniculata standardized to 5.6% of Andrographolide:Siberian Ginseng:American Ginseng:Vitamin C, in a ratio of 1:0.5-1.5:0.5-1.5:0.25-0.75. When the Andrographolide, Siberian Ginseng, American Ginseng, and Vitamin C (collectively ACTIVES) are in combination in specific ratios, they provide for a single dosage form that allows increased efficacious therapy to occur. There are many dosage forms known in the art, as detailed below. A preferred dosage form is a tablet.

In one embodiment the present invention comprises
An oral dosage form comprising;
   (a) Andrographis paniculata standardized to 5.6% of Andrographolide;
   (b) Siberian Ginseng;
   (c) American Ginseng; and
   (d) Vitamin C
in a ratio of (a):(b):(C):(d) of 1:0.75-1.5:0.75-1.5:0.25-0.75

In a preferred embodiment the dosage form is a single unit dosage form. In a preferred embodiment, Andrographis paniculata standardized to 5.6% of Andrographolide.

The dosage form may be any dosage form acceptable for delivery of a therapeutic substance to a patient. The compositions can be provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, or transmucosal, delivery, although oral delivery is presently preferred.

Most preferred is a dosage form that is a tablet or capsule.

In a preferred embodiment, the dosage form further comprises an enteric coating.

It is also preferred that the dosage form be provided as a single dosage unit.

It is an object of the present invention of the present invention to provide therapeutic levels of Andrographolide, Siberian Ginseng, American Ginseng, and Vitamin C in a single dosage form.

### DETAILED DESCRIPTION OF THE PREFERRED EMBODIMENTS

The present invention provides for a single unit dosage form delivering improved therapy of a combination of Andrographolide, Siberian Ginseng, American Ginseng, and Vitamin C.

The present invention has found an improved combination of therapy when using Andrographis paniculata (AP) extract standardized to 5.6% Andrographolide. As used herein, standardized extract will refer to will refer to Chasteberry extract standardized to 5.6% Andrographolide. Previous dosage forms have used AP extract in amounts up to 1200 mg per dose. The present invention has discovered the standardized AP extract provides enhanced therapeutic effects when administered in specific ratio combination with Siberian Ginseng, American Ginseng, and Vitamin C. Thus a unit dose of the present invention may be 100 mg or less.

The dose of Siberian Ginseng is may typically be 2-6 g/day. In a preferred embodiment, the present invention provides a unit dose of Siberian Ginseng of 100 mg.

American ginseng is typically administered in amounts between 1-2 g/day. In a preferred embodiment, the present invention provides a unit dose of American ginseng of 100 mg.

Vitamin C has a USDA recommended amount of 75-100/mg day. Vitamin C treatment regimens have long been debated with many scientists recommending dosage up to 1-4 g/day. In a preferred embodiment, the present invention provides a unit dose of Vitamin C of 50 mg

The compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art.

The composition can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition.

The term “enteric coating” as used herein relates to a mixture of pharmaceutically acceptable excipients that is applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Alternatively, an enteric coating may be applied in an aqueous/organic cosolvent system. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Cellulose Derivatives are a preferred enteric coat material. Examples of suitable cellulose derivatives are: ethyl cellulose; reaction mixtures of partial acetate esters of cellulose with phthalic anhydride.

A preferred coating is aqueous Ethylcellulose Dispersion. The dispersion is a combination of film-forming polymer; plasticizer and stabilizers. Designed for sustained release and taste masking applications, the dispersion provides the flexibility to adjust drug release rates with reproducible profiles that are relatively insensitive to pH.

The principal means of drug release is by diffusion through the dispersion membrane and is directly controlled by film thickness. Increasing or decreasing the quantity of dispersion applied can easily modify the rate of release.

Two well-known dispersions are Surelease (Colorcon, West Point, Pa.) and Aquacoat ECD (FMC).

The performance of a coating can vary based on the degree and type of substitution. Cellulose acetate phthalate (CAP) dissolves in pH>6. Aquateric (FMC) is an aqueous based system and is a spray dried CAP psuedolatex. Other components in Aquateric can include pluronics, Tweens, and acetylated monoglycerides; cellulose acetate trimellitate (Eastman); methylcellulose (Pharmacoat, Methocel); hydroxypropyl methyl cellulose phthalate (HPMCP). The performance can vary based on the degree and type of substitution. HP-50, HP-55, HP-55S, HP-55F grades are suitable; hydroxypropyl methyl cellulose succinate (HPMCS; AQOAT (Shin Etsu)).

The coating can, and usually does, contain a plasticizer and possibly other coating excipients such as colorants, talc, and/or magnesium stearate, which are well known in the art. Suitable plasticizers include: triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the lower intestinal tract is reached.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

A coating process frequently involves spraying a coating solution onto a substrate. The coating solution can be a molten solution of the encapsulation coat composition free of a dispersing, medium. The coating solution can also be prepared by solubilizing or suspending the composition of the encapsulation coat in an aqueous medium, an organic solvent, a supercritical fluid, or a mixture thereof. At the end of the coating process, the residual dispersing medium can be further removed to a desirable level utilizing appropriate drying processes, such as vacuum evaporation, heating, freeze drying, etc.

Solvent-based coating is when the components of the invention are solubilized and/or dispersed in a solvent. The solvent can be aqueous. When the solvent is aqueous-based, the components can be emulsified with an appropriate emulsifier, organic solvent, or a supercritical fluid. Solvents with a lower melting point than water and higher evaporation numbers are preferred. Solvent mixtures with other organic solvents or water are often employed to get appropriate viscosity and component solubilization. Typical solvents include ethanol, methanol, isopropanol, acetone, dichloromethane, trichloromethane and ethyl acetate. Appropriate polymers can also be added as needed. Cellulosic derivatives and polymethacrylates are particularly suitable additives for organic solvent coating. Dissolution and solubilization of the components is facilitated by rigorous stirring or heating. Plasticizers may be also be added to stimulate dissolution. Colorants and antisticking agents can be employed as needed.

The following are presented by way of example and are not intended to limit the scope of the invention.

One general formulation is as follows:

Andrographis Paniculata
standardized to 5.6% of

| | |
|---|---|
| Andrographolide | 1-25% |
| Siberian Ginseng | 1-25% |
| American Ginseng | 1-25% |
| Vitamin C | 1-15% |
| Filler | 20-75% |
| Binder | 1-20% |
| Disintegrant | up to 15% |
| Lubricant | up to 10% |
| Glident | up to 10% |

### Example 1

In one embodiment a first blend comprising 150.6 g AP Extract, 150.6 g Siberian Ginseng, 150.6 g American Ginseng, 75.3 g of Vitamin C and 100 g of microcrystalline cellulose (a common form sold as AVICEL® by FMC, Philadelphia, Pa.), are passed through a 25 mesh screen and blended until uniformly mixed. A second blend is prepared comprising 87.9 g microcrystalline cellulose 53.1 g stearic acid, and 33.2 g croscarmellose sodium are each passed through a 25 mesh screen. The first and second blends are combined in a v-blender and mixed 45 minutes or long enough to ensure content uniformity as is commonly known and practiced in the art. The blender is stopped and 6.6 mg of silicon dioxide and 33.2 mg of magnesium stearate are screened through a 25 mesh screen and added to the blender. The mixture is blended an additional five minutes. The tableting mixture is discharged from the blender. Capsule shaped tablets with a target weight of 664.2 mg (+/−6%) are compressed with a target hardness of 8-12 kP.

Tablets prepared according to Example 1 may optionally be coated with a layer. Alternatively, the tablets may be coated with more than one layer. Any layer may be functional or non-functional and may include, but would not be limited to controlled release, delayed release, sustained release, color, taste masking, moisture barrier, or any other layer disposed on the surface as are commonly practices in the art. In one embodiment, the tablets are coated with an enteric layer such that they do not dissolve in the gastric pH of approximately 1.2.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An oral dosage form comprising;
(a) Andrographis paniculata standardized to 5.6% of Andrographolide;
(b) Siberian Ginseng;
(c) American Ginseng; and
(d) Vitamin C
in a ratio of (a):(b):(C):(d) of 1:0.75-1.5:0.75-1.5:0.25-0.75.

2. The dosage form of claim 1 having a ratio of 1:0.8-1.2:0.8-1.2:0.35-0.65.

3. The dosage form of claim 1 wherein Vitamin C is present as a salt, acid, isomer, or derivative.

4. The dosage form of claim 1 wherein Vitamin C is present as L-ascorbate.

5. The dosage form of claim 1 wherein said dosage form is a tablet or capsule.

6. The dosage form of claim 1 wherein said dosage form further comprises an enteric coating.

7. The dosage form of claim 1 wherein said dosage form is provided as a single unit.

8. The dosage form of claim 1 comprising:
(a) Andrographis paniculata standardized to 5.6% of Andrographolide 1-25%;
(b) Siberian Ginseng 1-25%;
(c) American Ginseng 1-25%; and
(d) Vitamin C 1-15%
based on the total weight of the dosage form.

9. The dosage form of claim 1 comprising a coating disposed on said dosage form.

10. The dosage form of claim 9 wherein said coating is at least one layer.

11. The dosage form of claim 9 wherein said layer is functional or non-functional.

12. The dosage form of claim 1 comprising a functional layer selected from controlled release, sustained release, delayed release, taste masking, or moisture control.

13. The dosage form of claim 1 comprising an enteric layer.

14. A method of providing therapy to a patient comprising or consisting of the steps of:
(a) preparing a single unit dosage form comprising
(i) Andrographis paniculata standardized to 5.6% of Andrographolide, 1-25%;
(ii) Siberian Ginseng 1-25%;
(iii) American Ginseng 1-25%; and
(iv) Vitamin C 1-15%
said Andrographis paniculata, Siberian Ginseng, American Ginseng, and Vitamin C being present in a ratio of 1:0.75-1.5:0.75-1.5:0.25-0.75 and being present in any form including salts, isomers, and derivatives;
(b) administering said single unit dosage form to a patient.

15. A method for providing therapy to treat, prevent or ameliorate colds, flu and related disorders, comprising or consisting of the steps of:
(a) preparing a single unit dosage form comprising
(i) Andrographis paniculata standardized to 5.6% of Andrographolide, 1-25%;
(ii) Siberian Ginseng 1-25%;
(iii) American Ginseng 1-25%; and
(iv) Vitamin C 1-15%
said Andrographis paniculata, Siberian Ginseng, American Ginseng, and Vitamin C being present in a ratio of 1:0.75-1.5:0.75-1.5:0.25-0.75 and being present in any form including salts, isomers, and derivatives;
(c) administering said single unit dosage form to a patient.
